(19) United States
(12) Patent Application Publication
(10) Pub. No.: US 2022/0271575 A1
(43) Pub. Date: Aug. 25, 2022

(54) LEARNING ALGORITHM FOR RECHARGE SYSTEM

(71) Applicant: Medtronic, Inc., Minneapolis, MN (US)

(72) Inventors: Andrew Thomas Fried, St. Paul, MN (US); Charles M. Nowell, Longwood, FL (US); Andrew L. Schmeling, Holmen, WI (US); Reid K. Bornhoft, Lino Lakes, MN (US); Tressa Marie Norden, New Berlin, WI (US); Pooja Raghavan, St. Louis Park, MN (US)

(21) Appl. No.: 17/652,241

(22) Filed: Feb. 23, 2022

Related U.S. Application Data
(60) Provisional application No. 63/153,319, filed on Feb. 24, 2021.

(51) Int. Cl. H02J 50/90; A61N 1/378; H02J 50/23; H02J 50/10; H02J 13/00; H02J 7/00

(52) U.S. Cl. CPC H02J 50/90; A61N 1/3787; H02J 50/23; H02J 50/10; H02J 13/00001; H02J 7/0071; H02J 2310/23; A61N 1/36062

(57) ABSTRACT

Devices, systems, and techniques are described to provide consistent power transfer from a power transmitting unit to power receiving unit. In an example of recharging an electrical energy storage device, e.g., a battery, consistent power transfer may result in consistent recharge durations. A system may include a training mode in which a user may change a location of the power transfer unit relative to the power receiving unit. The system may provide an output to the user with a relative for a consistent power transfer. In other examples, a power transfer system may include a learning algorithm that measures and stores the power transfer during power transfer for a number of sessions over time. The learning algorithm may provide an output to a user of a relative location and/or relative orientation of the power transfer unit and power receiving unit that provides a consistent power transfer.

This application claims the benefit of U.S. Provisional Patent Application No. 63/153,319, filed Feb. 24, 2021, the entire contents of which is incorporated herein by reference.

### TECHNICAL FIELD

The disclosure relates wireless power transfer, and particularly to power transfer for recharging an electrical energy storage device.

### BACKGROUND

Power may be transmitted wirelessly from a power transmitting unit (PTU) to a power receiving unit (PRU) for example by transmitting radio frequency (RF) energy, by inductive coupling and so on. In some examples the PTU and PRU may also communicate, e.g., send digital messages back and forth, using RF communication or inductive communication before, during or after transferring power. In some examples, the PTU may wirelessly transfer energy to the PRU to recharge, for example, a battery, a storage capacitor or some other electrical energy storage device in the PRU. In some examples, the power receiving unit may be an implantable medical device configured to receive electrical energy via transcutaneous power transfer.

### SUMMARY

In general, the disclosure describes devices, systems, and techniques that provide consistent power transfer from a power transmitting unit to power receiving unit. In the example of recharging an electrical energy storage device, such as a battery, consistent power transfer may result in consistent recharge durations. For example, a consistent recharge duration may be approximately one hour rather than a half-hour for some recharging sessions and several hours for other recharging sessions when using the same power transmitting unit and power receiving unit. In some examples, a power transfer system of this disclosure, e.g., a power transfer unit and power receiving unit, may include a training mode. During training mode, a user may change a location and orientation of the power transfer unit relative to the power receiving unit. The power transfer system may determine the location and/or orientation that provides a consistent power transfer and outputs an indication to the user, e.g., via a user interface, this location and/or orientation.

In other examples, a power transfer system may include a learning algorithm, e.g., executed by processing circuitry, that measures and stores the power transfer during power transfer sessions over time. In some examples the learning algorithm may record coupling efficiency, or some other measure of power transfer, for a number of power transfer sessions. The learning algorithm may determine an average, median or some other measure of the power transfer and provide an output to a user of a relative location and/or relative orientation of the power transfer unit and power receiving unit that provides a consistent power transfer.

In another example, this disclosure describes a system comprising a user interface; power transfer measurement circuitry; a power transmitting circuit comprising a transmit antenna configured to transmit electromagnetic energy to a power receiving device; processing circuitry operatively coupled to a memory, the processing circuitry configured to: control the power transmitting circuit to wireles sly output the electromagnetic energy to the power receiving device; receive, from the power transfer measurement circuit, an indication of an amount of power transferred to the power receiving device; record a plurality of power transfer measurements; and control the user interface to output an indication of the amount of power transferred, wherein the indication of the amount of power transferred is configured to prompt a user to adjust a position of the transmit antenna relative to the power receiving device based on the plurality of power transfer measurements.

In another example, this disclosure describes a system comprising a user interface; a power transfer measurement circuit; a power transmitting circuit comprising a transmit antenna; processing circuitry operatively coupled to a memory, the processing circuitry configured to: control the power transmitting circuit to wirelessly output electromagnetic energy; receive from the power transfer measurement circuit an indication of an amount of power transferred to a power receiving unit (PRU); during a power transfer session, record a plurality of power transfer efficiency measurements; determine a session power transfer efficiency value based on a first measure of central tendency for the plurality of power transfer efficiency measurements; determine a system power transfer efficiency based on a second measure of central tendency for a plurality of session power transfer efficiency values; calculate a threshold power transfer efficiency based on the system power transfer efficiency; and output an indication via the user interface of a relative location between the transmit antenna and the power receiving unit that provides a session power transfer efficiency above the threshold power transfer efficiency.

In another example, this disclosure describes a method comprising controlling, by processing circuitry operatively coupled to a memory, a power transmitting circuit to wireles sly output electromagnetic energy to power receiving device, wherein the power transmitting circuit comprises a transmit antenna configured to output the electromagnetic energy to the power receiving device; receiving, by the processing circuitry and from a power transfer measurement circuit, an indication of an amount of power transferred to the power receiving device; recording, by the processing circuitry, a plurality of power transfer measurements; controlling, by the processing circuitry, a user interface to output an indication of the amount of power transferred, wherein the indication of the amount of power transferred is configured to prompt a user to adjust a position of the transmit antenna relative to the power receiving device based on the plurality of power transfer measurements.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example system that includes a power receiving unit and an external charging device that transfers power to the power receiving unit in accordance with the techniques described in this disclosure.

FIG. 2 is a block diagram of the example power receiving unit of FIG. 1.

FIG. 3 is a block diagram of the example external charging device of FIG. 1.

FIGS. 4A and 4B are a conceptual diagrams illustrating example user interfaces that display example movement patterns for a power transmit unit antenna relative to a power receiving unit during a training mode.

FIG. 5 is a conceptual diagram illustrating an example user interface display of a training mode of a power transmit unit according to one or more techniques of this disclosure.

FIG. 6 is a conceptual diagram illustrating an example user interface peak signal display of a training mode of a power transmit unit according to one or more techniques of this disclosure.

FIGS. 7A and 7B are a conceptual diagram illustrating an example user interface user criteria selection screen for a power transmit unit according to one or more techniques of this disclosure.

FIG. 8 is a flow diagram illustrating an example operation of the power transfer system of this disclosure.

### DETAILED DESCRIPTION

The disclosure describes devices, systems, and techniques that provide consistent power transfer from a power transmitting unit to power receiving unit. The power transfer efficiency may change based on the relative position of the power receiving unit to the power transmitting unit. In the example of recharging an electrical energy storage device, such as a battery, consistent power transfer may result in consistent recharge durations. In some examples, a power transfer system of this disclosure, e.g., a system that includes a power transfer unit (PTU) and power receiving unit (PRU), may include a training mode. During training mode, a user may change a location and orientation of the power transfer unit relative to the power receiving unit, in some examples, based on a specified pattern. The power transfer system may determine the location and or orientation that provides a consistent power transfer and output an indication, e.g., via a user interface to the user. A user interface may include an audio output, e.g., a tone that changes tone, frequency, pulse repetition or some other audio characteristic as the user changes the relative location or orientation. A user interface may also include a display that changes color, length of a bar on a bar chart, a moving needle or some other indication of power transfer.

In other examples, a power transfer system of this disclosure may include a learning algorithm, e.g., executed by processing circuitry, that measures and stores the power transfer during power transfer sessions over time. In some examples the learning algorithm may record coupling efficiency, or some other measure of power transfer, over a number of recording sessions. Over time, the learning algorithm may determine a measure of central tendency, such as an average, median or some other measure of the power transfer and output an indication to a user describing a relative location and/or relative orientation of the power transfer unit and power receiving unit that provides a consistent power transfer. In this manner, the system can train the user on how to efficiently charge the system.

As noted above, inconsistencies in power transfer may be caused by changes in the orientation and location of power transmitting unit relative to the power receiving unit. In some examples a power transmitting unit may include a transmit antenna, e.g., a transmitting coil in the example of an inductive power transmitting unit. Similarly, the power receiving unit may include a receive antenna. In some examples, for an inductive system, the transmit coil parallel to the receive coil may maximize coupling efficiency. As the relative angle changes, the coupling efficiency may change. Examples of changes in location may include a distance between the power transmitting unit and power receiving unit, or more particularly, the distance between the power transmit antenna and receiving antenna. Other factors that may impact the power transfer may include material between the power transmitting unit and power receiving unit that may absorb the wireless energy, manufacturing differences or model to model differences from unit to unit in power transmitting devices and power receiving devices, as well as other factors.

By configuring the power transfer system based on individual characteristics of the power transmitting unit and the power receiving unit, the techniques of this disclosure may provide advantages over other types of power transfer systems. Some examples of power transfer systems are pre-configured based on estimates for a particular type of power transfer unit and type of power receiving unit. However, the configuration of the power transfer system may be based on estimates for a nominal transmitter and nominal receiver and may not be individually configured for a particular situation. Such pre-configured systems may not provide consistent power transfer. In some examples, the techniques of this disclosure factor in the specific power transfer for a specific power transfer unit with a specific power receiving unit in a specific environment to provide consistent power transfer as well as may provide a more accurate estimate of how long charging may take.

In the example of a power receiving unit that is an implantable medical device, the same model of device may be implanted in different locations from patient to patient, depending on the patient's condition, anatomy and other factors result in different power transfer environment. The different power transfer environment may cause differences in power transfer from patient to patient.

In some examples, same patient may gain or lose weight, which may increase or decrease the adipose tissue (body fat) between the implanted medical device and the power transmitting unit. The increase or decrease may change the environment and therefore the amount of energy absorbed by the patient's tissue, as well as the relative distance between the power transmitting unit and implanted medical device. For a power transfer system including a medical device, the “user” may include the patient, a clinician treating the patient, or some other caregiver, e.g., a family member or in-home health care.

In the example of a patient with a rechargeable medical device, a consistent recharge duration may be desirable to be able to plan around the patient's daily activities. As noted above, a recharge duration may be approximately one hour rather than a half-hour for some recharging sessions and several hours for other recharging sessions when using the same power transmitting unit and power receiving unit. In some examples, the patient may prefer easier power transfer coupling rather than consistent recharge times.

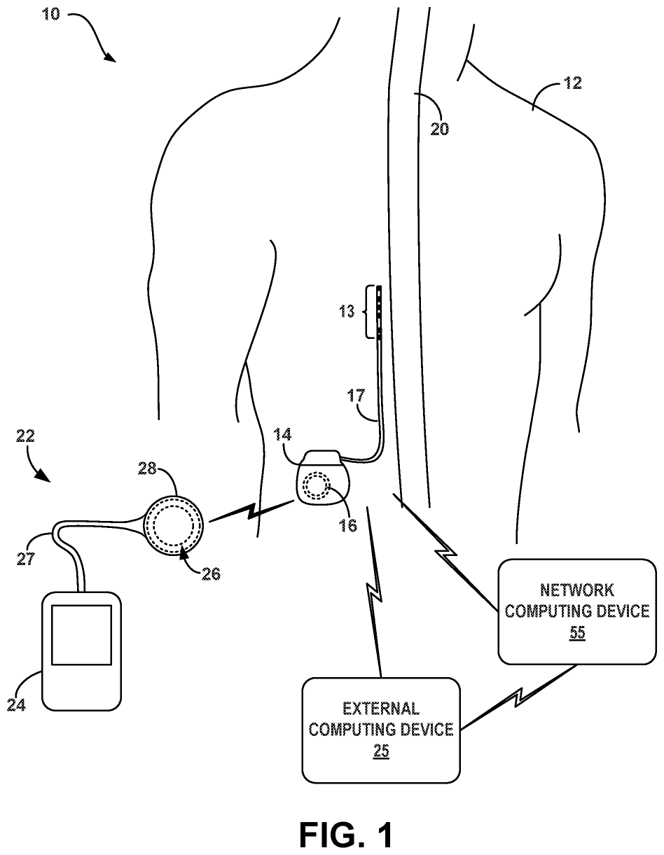

FIG. 1 is a conceptual diagram illustrating example system 10 that includes an implantable medical device (IMD) 14 and an external charging device 22 that charges a rechargeable power source of the IMD 14 via an energy transfer coil 26. Although the techniques described in this disclosure are generally applicable to a variety of devices including medical devices such as patient monitors, electrical stimulators, or drug delivery devices, application of such techniques to implantable neurostimulators will be described for purposes of illustration. More particularly, the disclosure will refer to an implantable neurostimulation system for use in spinal cord stimulation therapy, but without limitation as to other types of medical devices. In some examples IMD 14 may also be referred to as implantable neurostimulator (INS) 14.

As shown in FIG. 1, system 10 includes an IMD 14 and external charging device 22 shown in conjunction with a patient 12, who is ordinarily a human patient. In the example of FIG. 1, IMD 14 is an implantable electrical stimulator that delivers neurostimulation therapy to patient 12, e.g., for relief of chronic pain or other symptoms. Generally, IMD 14 may be a chronic electrical stimulator that remains implanted within patient 12 for weeks, months, or even years. In the example of FIG. 1, IMD 14 and lead 17 placed near spinal cord 20 and may be directed to delivering spinal cord stimulation therapy. In other examples, IMD 14 may be a temporary, or trial, stimulator used to screen or evaluate the efficacy of electrical stimulation for chronic therapy. IMD 14 may be implanted in a subcutaneous tissue pocket, within one or more layers of muscle, or other internal location. IMD 14 includes a rechargeable power source (not shown) and IMD 14 is coupled to lead 17.

System 100 may also include network computing device 55 configured to communicate with the external computing device 25 and/or directly with IMD 14. Network computing device may be a network server, e.g., a cloud server, a local server in the home of the patient, or in the office of a caregiver. In other examples, network computing device 55 may be a laptop computer, mobile smart phone, tablet computer or other computing device which may comprise processing circuitry, computer readable storage media, a user interface and other similar components. The functions described for system 100 may be programming instructions executed by any one or any combination of processing circuitry in IMD 14, external computing device 25 and network computing device 55.

Electrical stimulation energy, which may be constant current or constant voltage based pulses, for example, is delivered from IMD 14 to one or more targeted locations within patient 12 via one or more electrodes 13 of lead 17. The parameters for a program that controls delivery of stimulation energy by IMD 14 may include information identifying which electrodes of electrodes 13 that have been selected for delivery of stimulation according to a stimulation program, the polarities of the selected electrodes, i.e., the electrode configuration for the program, and voltage or current amplitude, pulse rate, pulse shape, and pulse width of stimulation delivered by the electrodes. Electrical stimulation may be delivered in the form of stimulation pulses or continuous waveforms, for example. In some examples, IMD 14 may be configured to monitor patient biological signals, such as biological impedance, cardiac signals, temperature, activity, and so on. In some examples IMD 14 may not deliver stimulation therapy.

In the example of FIG. 1, lead 17 is disposed within patient 12, e.g., implanted within patient 12. Lead 17 tunnels through tissue of patient 12 from along spinal cord 20 to a subcutaneous tissue pocket or other internal location where IMD 14 is disposed. Although lead 17 may be a single lead, lead 17 may include a lead extension or other segments that may aid in implantation or positioning of lead 17. In addition, a proximal end of lead 17 may include a connector (not shown) that electrically couples to a header of IMD 14. Although only one lead 17 is shown in FIG. 1, system 10 may include two or more leads, each coupled to IMD 14 and directed to similar or different target tissue sites. For example, multiple leads may be disposed along spinal cord 20 or leads may be directed to spinal cord 20 and/or other locations within patient 12. Lead 17 may carry one or more electrodes 13 that are placed adjacent to the target tissue, e.g., spinal cord 20 for spinal cord stimulation (SCS) therapy.

In alternative examples, lead 17 may be configured to deliver stimulation energy generated by IMD 14 to stimulate one or more sacral nerves of patient 12, e.g., sacral nerve stimulation (SNS). SNS may be used to treat patients suffering from any number of pelvic floor disorders such as pain, urinary incontinence, fecal incontinence, sexual dysfunction, or other disorders treatable by targeting one or more sacral nerves. Lead 17 and IMD 14 may also be configured to provide other types of electrical stimulation or drug therapy (e.g., with lead 17 configured as a catheter). For example, lead 17 may be configured to provide deep brain stimulation (DBS), peripheral nerve stimulation (PNS), or other deep tissue or superficial types of electrical stimulation. In other examples, lead 17 may provide one or more sensors configured to allow IMD 14 to monitor one or more biological signals of patient 12. The one or more sensors may be provided in addition to, or in place of, therapy delivery by lead 17.

IMD 14 delivers electrical stimulation therapy to patient 12 via selected combinations of electrodes carried by lead 17. The target tissue for the electrical stimulation therapy may be any tissue affected by electrical stimulation energy, which may be in the form of electrical stimulation pulses or waveforms. In some examples, the target tissue includes nerves, smooth muscle, and skeletal muscle. In the example illustrated by FIG. 1, the target tissue for electrical stimulation delivered via lead 17 is tissue proximate spinal cord 20 (e.g., one or more target locations of the dorsal columns or one or more dorsal roots that branch form spinal cord 20. Lead 17 may be introduced into spinal cord 20 via any suitable region, such as the thoracic, cervical or lumbar regions. Stimulation of dorsal columns, dorsal roots, and/or peripheral nerves may, for example, prevent pain signals from traveling through spinal cord 20 and to the brain of the patient. Patient 12 may perceive the interruption of pain signals as a reduction in pain and, therefore, efficacious therapy results. For treatment of other disorders, lead 17 may be introduced at any exterior location of patient 12.

Although lead 17 is described as generally delivering or transmitting electrical stimulation signals, lead 17 may additionally or alternatively transmit bioelectrical signals from patient 12 to IMD 14 for monitoring. For example, IMD 14 may utilize detected nerve impulses to diagnose the condition of patient 12 or adjust the delivered stimulation therapy. Lead 17 may thus transmit electrical signals to and from patient 12.

A user, such as a clinician or patient 12, may interact with a user interface of an external computing device 25 to communicate with and in some examples, to program IMD 14. Programming of IMD 14 may refer generally to the generation and transfer of commands, programs, or other information to control the operation of IMD 14. For example, the external programmer may transmit programs, parameter adjustments, program selections, group selections, or other information to control the operation of IMD 14, e.g., by wireless telemetry or wired connection.

In some cases, external computing device 25 may be characterized as a physician or clinician programmer if it is primarily intended for use by a physician or clinician. In other cases, external computing device 25 may be characterized as a patient programmer if it is primarily intended for use by a patient. A patient programmer is generally accessible to patient 12 and, in many cases, may be a portable device that may accompany the patient throughout the patient's daily routine. In general, a physician or clinician programmer may support selection and generation of programs by a clinician for use by stimulator 14, whereas a patient programmer may support adjustment and selection of such programs by a patient during ordinary use. In other examples, external charging device 22 may be included, or part of, an external programmer. In this manner, a user may program and charge IMD 14 using one device, or multiple devices.

IMD 14 may be constructed of any polymer, metal, or composite material sufficient to house the components of IMD 14 within patient 12. In this example, IMD 14 may be constructed with a biocompatible housing, such as titanium or stainless steel, or a polymeric material such as silicone or polyurethane, and surgically implanted at a site in patient 12 near the pelvis, abdomen, or buttocks. The housing of IMD 12 may be configured to provide a hermetic seal for components, such as a rechargeable power source. In addition, the housing of IMD 12 may be selected of a material that facilitates receiving energy to charge a rechargeable power source.

As described herein, secondary coil 16 may be included within IMD 14. However, in other examples, secondary coil 16 could be located external to a housing of IMD 14, separately protected from fluids of patient 12, and electrically coupled to electrical components of IMD 14. This type of configuration of IMD 14 and secondary coil 16 may provide implant location flexibility when anatomical space available for implantable devices is minimal and/or improved inductive coupling between secondary coil 16 and primary coil 26. In any case, an electrical current may be induced within secondary coil 16 to charge the battery of IMD 14 when energy transfer coil 26 (e.g., a primary coil) produces a magnetic field that is aligned with secondary coil 16. The induced electrical current may first be conditioned and converted by a charging module (e.g., a charging circuit) to an electrical signal that can be applied to the battery with an appropriate charging current. For example, the inductive current may be an alternating current that is rectified to produce a direct current suitable for charging the battery. In some examples, primary coil 26 may comprise multiple separate coils that are displaced in location from each other.

The rechargeable power source of IMD 14 may include one or more capacitors, batteries, or components (e.g., chemical or electrical energy storage devices). Example batteries may include lithium-based batteries, nickel metal-hydride batteries, or other materials. The rechargeable power source may be replenished, refilled, or otherwise capable of increasing the amount of energy stored after energy has been depleted. The energy received from secondary coil 16 may be conditioned and/or transformed by a charging circuit. The charging circuit may then send an electrical signal used to charge the rechargeable power source when the power source is fully depleted or only partially depleted.

Charging device 22 may be used to recharge the rechargeable power source within IMD 14 implanted in patient 12. Charging device 22 may be a hand-held device, a portable device, or a stationary charging system. In any case, charging device 22 may include components necessary to charge IMD 14 through tissue of patient 12. Charging device 22 may include housing 24 and energy transfer coil 26. In addition, heat sink device 28 may be removably attached to energy transfer coil 26 to manage the temperature of then energy transfer coil during charging sessions. Housing 24 may enclose operational components such as a processing circuitry 50, memory, user interface 54, telemetry circuitry 56, power source, and charging circuit configured to transmit energy to secondary coil 16 via energy transfer coil 26. Although a user may control the recharging process with a user interface of charging device 22, charging device 22 may alternatively be controlled by another device (e.g., network computing device 55). In other examples, charging device 22 may be integrated with an external programmer, such as a patient programmer carried by patient 12.

Charging device 22 and IMD 14 may utilize any wireless power transfer techniques that are capable of recharging the power source of IMD 14 when IMD 14 is implanted within patient 14. In one example, system 10 may utilize inductive coupling between primary coils (e.g., energy transfer coil 26) and secondary coils (e.g., secondary coil 16) of charging device 22 and IMD 14. In inductive coupling, energy transfer coil 26 is placed near implanted IMD 14 such that energy transfer coil 26 is aligned with secondary coil 16 of IMD 14. Charging device 22 may then generate an electrical current in energy transfer coil 26 based on a selected power level for charging the rechargeable power source of IMD 14. When the primary and secondary coils are aligned, the electrical current in the primary coil may magnetically induce an electrical current in the secondary coil within IMD 14. Since the secondary coil is associated with and electrically coupled to the rechargeable power source, the induced electrical current may be used to increase the voltage, or charge level, of the rechargeable power source. Although inductive coupling is generally described herein, any type of wireless energy transfer may be used to transfer energy between charging device 22 and IMD 14.

Energy transfer coil 26 may include a wound wire (e.g., a coil) (not shown in FIG. 1). The coil may be constructed of a wire wound in an in-plane spiral (e.g., a disk-shaped coil). In some examples, this single or even multi-layers spiral of wire may be considered a flexible coil capable of deforming to conform with a non-planar skin surface. The coil may include wires that electrically couple the flexible coil to a power source and a charging module configured to generate an electrical current within the coil. Energy transfer coil 26 may also include a housing that encases the coil. The housing may be constructed of a flexible material such that the housing promotes, or does not inhibit, flexibility of the coil. Energy transfer coil 26 may be external of housing 24 such that energy transfer coil 26 can be placed on the skin of patient 12 proximal to IMD 14. In this manner, energy transfer coil 26 may be tethered to housing 24 using cable 27 or other connector that may be between approximately a few inches and several feet in length. In other examples, energy transfer coil 26 may be disposed on the outside of housing 24 or even within housing 24. Energy transfer coil 26 may thus not be tethered to housing 22 in other examples.

Heat sink device 28 may be removably attached to energy transfer coil 26. In examples where energy transfer coil 26 is disposed on or within housing 24, heat sink device 28 may be configured to be removably attached to housing 24. In the example of system 10, charging device 22 is the power transmitting unit and IMD 14 is the power receiving unit. IMD 14 may be in a flipped or non-flipped position.

Heat sink device 28 may include a housing that contains a phase change material. The housing may be configured to be removably attached to energy transfer coil 26. In this manner, the system may operate such that energy transfer coil 26 generates heat during a recharge session and the phase change material of heat sink device 28 absorbs at least a portion of the generated heat. When the phase change material is at the melting temperature, the heat may contribute to the heat of fusion of the phase change material and not to increasing the temperature of energy transfer coil 26.

In some examples, energy transfer coil 26 may implemented as a flexible coil configured to conform to a surface, such as an ankle of patient 12 in the example in which IMD 14 is implanted for tibial stimulation. As a flexible coil, energy transfer coil 26 may be formed by one or more coils of wire. In one example the coil is formed by a wire wound into a spiral within a single plane (e.g., an in-plane spiral). This in-plane spiral may be constructed with a thickness equal to the thickness of the wire, and the in-plane spiral may be capable of transferring energy with another coil. In other examples, the coil may be formed by winding a coil into a spiral bent into a circle. However, this type of coil may not be as thin as the in-plane spiral.

Based on changes in the relative position of the primary coil 26 and secondary coil 16, a charging system may deliver inconsistent recharge time. In some examples external charging device 22 may include a training mode on to help improve the coupling position between the primary coil and the power receiving unit, e.g., IMD 14. The training mode may include a display on a user interface for users to calibrate the power transmitting unit, e.g., external charging device 22, to find a relative position for high power transfer for coupling. Processing circuitry, which may be located as part of external charging device 22, external device 25 or IMD 14, may execute the training mode from a closed loop recharge session where the external charging device 22 and IMD 14 are connected.

Training mode may include a variety of options to determine the best coupling for each individual patient, e.g., processing circuitry may execute a different few factors and sequences. First, the user interface may ask the patient to move their primary coil 26 around to different locations and hold at various positions around the site of IMD 14. The processing circuitry may determine the coupling strength and/or efficiency at each location. The processing circuitry may determine an average coupling efficiency, or other measure of central tendency for all the different relative positions. The “average_coupling_eff” may be a system metric based on Pins_batt/Ptank (total efficiency), Pins_batt/Q_{INS} (INS or IMD efficiency), Pins_batt/Pins or it could be another system metric such as INS battery current or metal loading. Pins is the power sent to the power receiving device, e.g., to IMD 14. Pins_batt is the power received by the battery of IMD 14, which may be measured by battery current, or a similar measurement, and may be communicated to external charging device 22 by IMD 14, in some examples. Qins is the amount of heat lost, e.g., absorbed by the surrounding tissue or by the circuitry of IMD 14. Qins may be found from:

Pᵢₙₛ=P_{ins_batt}+Qᵢₙₛ

For example: This individual patient's “excellent threshold” for charging may be set to: 0.9*max(average_coupling_eff[1−N]), and this individual patient's “good threshold” for charging could then be: 0.75*max(average_coupling_eff[1−N]). Furthermore, the threshold value e.g., of 0.9 or 0.75 could be adjusted by the user via a user interface of charging system 22, or some other external computing device, such as network computing device 55. For example, the user may select 0.85 as the “excellent threshold” instead of 0.9. The user interface may also provide the patient options to choose between large sweet spot or consistent recharge time. In this disclosure the “sweet spot” may be a relative location between the primary coil, e.g., energy transfer coil 26, and secondary coil 16 in which the power transfer is above a specified threshold, based on one or more system metrics. Relative locations outside the “sweet spot” may result in power transfer less than the specified threshold.

In this disclosure, Qins may be a calculated estimate of the amount of heating of the implantable medical device, as noted above. P_{TANK}, is a calculated value for the power sent to primary coil 48 of energy transfer coil 26, and may include the inductance and capacitance between power generation circuitry, e.g., charging circuitry 56 and primary coil 48. Pins_batt is the amount of power delivered to the electrical energy storage device, e.g., power source 18 of IMD 14, as described above.

During the training mode, for each location the patient moves the wireless recharger to, a measurement will be taken to determine the amount of metal detected, efficiency, current or other similar system metrics. This information will be stored, so once all measurements are taken, the best coupling for the patient can be determined. The user interface may then guide the patient back to that spot. In some examples, the user interface may describe different patterns that the user should follow to move the primary coil. In some examples, the user interface may provide an indication of where the primary coil is in a selected pattern, relative to the power receiving unit, or some other location indicator. For example, the primary coil may be displayed as a circle that grows larger or smaller based on relative location. In some examples, the indication displayed by the user interface may recommend a positional adjustment to the user.

In some examples, processing circuitry of system 100 may execute instructions for a flow in the state machine to train the recharger to find an “excellent” coupling position. For example, the programming instructions may control the recharger to calculate recharge status on a more regular basis (every second, for example) when it is already in an open telemetry session. Over time, such as if the patient gains weight or IMD 14 moves in the implant pocket, the user can initiate training again to find new “excellent” position/level.

In this way, people with large excellent coupling areas (shallow implants) can have higher consistency recharge time or people with deep implants can have greater ease of achieving coupling. This feature can make rechargeable easier to use and charging time more consistent and may provide a way to individually tailor the recharge system to each patient/implant, which may be an advantage of the system of this disclosure, when compared to other power transfer systems.

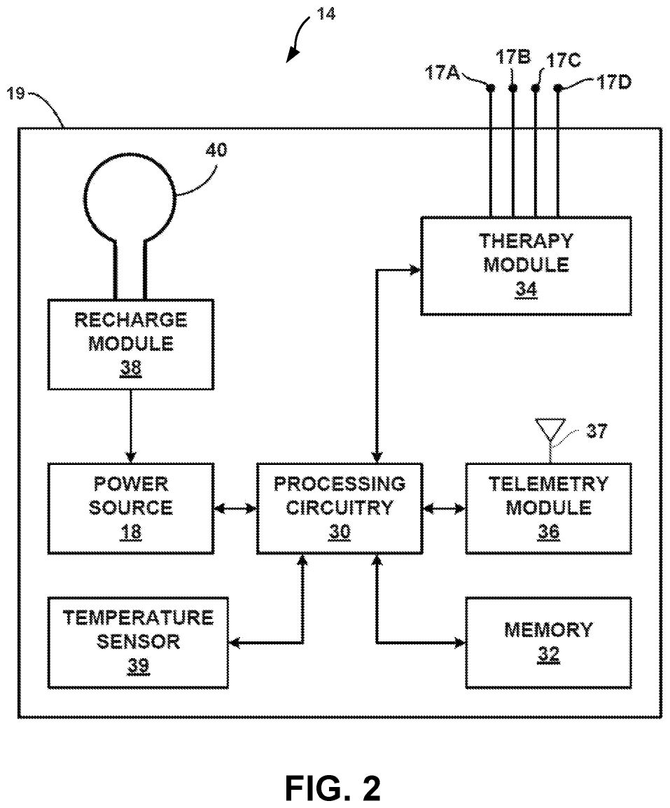

FIG. 2 is a block diagram illustrating example components of IMD 14 of FIG. 1. In the example illustrated in FIG. 2, IMD 14 includes temperature sensor 39, coil 40, processing circuitry 30, therapy module 34, recharge module 38, memory 32, telemetry module 36, and rechargeable power source 18. In other examples, IMD 14 may include a greater or a fewer number of components. In general, IMD 14 may comprise any suitable arrangement of hardware, alone or in combination with software and/or firmware, to perform the various techniques described herein attributed to IMD 14 and processing circuitry 30, and any equivalents thereof.

Processing circuitry 30 of IMD 14 may include one or more processors, such as one or more microprocessors, digital signal processors (DSPs), application specific integrated circuits (ASICs), field programmable gate arrays (FPGAs), or any other equivalent integrated or discrete logic circuitry, as well as any combinations of such components. IMD 14 may include a computer readable storage media, e.g., memory 32, such as random access memory (RAM), read only memory (ROM), programmable read only memory (PROM), erasable programmable read only memory (EPROM), electronically erasable programmable read only memory (EEPROM), flash memory, comprising executable instructions for causing the processing circuitry 30 to perform the actions attributed to this circuitry. Moreover, although processing circuitry 30, therapy module 34, recharge module 38, telemetry module 36, and temperature sensor 39 are described as separate modules, in some examples, some combination of processing circuitry 30, therapy module 34, recharge module 38, telemetry module 36 and temperature sensor 39 are functionally integrated. In some examples, processing circuitry 30, therapy module 34, recharge module 38, telemetry module 36, and temperature sensor 39 correspond to individual hardware units, such as ASICs, DSPs, FPGAs, or other hardware units.

Memory 32 may store therapy programs or other programming instructions that when executed by processing circuitry 30, specify therapy parameter values for the therapy provided by therapy module 34 and IMD 14. In some examples, memory 32 may also store temperature data from temperature sensor 39, instructions for recharging rechargeable power source 18, thresholds, instructions for communication between IMD 14 and external charging device 22, or any other instructions required to perform tasks attributed to IMD 14. Memory 32 may be configured to store instructions for communication with and/or controlling one or more temperature sensors of temperature sensor 39. In various examples, memory 32 stores information related to determining the temperature of housing 19 and/or exterior surface(s) of housing 19 of IMD 14 based on temperatures sensed by one or more temperature sensors, such as temperature sensor 39, located within IMD 14.

For example, memory 32 may store one or more formulas, that may be used to determine system metrics, including the temperature of the housing 19 and/or exterior surface(s) of housing 19 based on temperature(s) sensed by the temperature sensor 39. Memory 32 may store values for one or more determined constants used by these formulas. Memory 32 may store instructions that, when executed by processing circuitry such as processing circuitry 30, perform an algorithm, including using the formulas, to determine a current temperature, or temperatures over time, for the housing 19 and/or exterior surface(s) of the housing 19 of IMD 14 during a charging session and/or for some time after a charging session performed on IMD 14, power transfer efficiency, or other system metrics. In some examples, memory 32 may store instructions that, when executed by processing circuitry such as processing circuitry 30, perform an algorithm, including using one or more formulas, to determine a value to be assigned to one or more of the constants used in the algorithm to determine a temperature for the housing 19 and/or exterior surface(s) of the housing 19 of IMD 14 during a charging session and/or for some time after a charging session performed on IMD 14.

Generally, therapy module 34 may generate and deliver electrical stimulation under the control of processing circuitry 30. In some examples, processing circuitry 30 controls therapy module 34 by accessing memory 32 to selectively access and load at least one of the stimulation programs to therapy module 34. For example, in operation, processing circuitry 30 may access memory 32 to load one of the stimulation programs to therapy module 34. In such examples, relevant stimulation parameters may include a voltage amplitude, a current amplitude, a pulse rate, a pulse width, a duty cycle, or the combination of electrodes 17A, 17B, 17C, and 17D (collectively “electrodes 17”) that therapy module 34 uses to deliver the electrical stimulation signal. Therapy module 34 may be configured to generate and deliver electrical stimulation therapy via one or more of electrodes 17A, 17B, 17C, and 17D of lead 16. Alternatively, or additionally, therapy module 34 may be configured to provide different therapy to patient 12. For example, therapy module 34 may be configured to deliver drug delivery therapy via a catheter. These and other therapies may be provided by IMD 14.

IMD 14 also includes components to receive power from external charging device 22 to recharge rechargeable power source 18 when rechargeable power source 18 has been at least partially depleted. As shown in FIG. 2, IMD 14 includes secondary coil 40 and recharge module 38 coupled to rechargeable power source 18. Recharge module 38 may be configured to charge rechargeable power source 18 with the selected power level determined by either processing circuitry 30 or external charging device 22. Recharge module 38 may include any of a variety of charging and/or control circuitry configured to process or convert current induced in coil 40 into charging current to charge power source 18. Although processing circuitry 30 may provide some commands to recharge module 38, in some examples, processing circuitry 30 may not need to control any aspect of recharging.

Secondary coil 40 may include a coil of wire or other device capable of inductive coupling with a primary coil disposed external to patient 12. Although secondary coil 40 is illustrated as a simple loop of in FIG. 2, secondary coil 40 may include multiple turns of conductive wire. Secondary coil 40 may include a winding of wire configured such that an electrical current can be induced within secondary coil 40 from a magnetic field. The induced electrical current may then be used to recharge rechargeable power source 18. In this manner, the electrical current may be induced in secondary coil 40 associated with rechargeable power source 18. The induction may be caused by electrical current generated in the primary coil of external charging device 22, where the level of the current may be based on the selected power level. The coupling between secondary coil 40 and the primary coil of external charging device 22 may be dependent upon the alignment of the two coils. Generally, the coupling efficiency increases when the two coils share a common axis and are in close proximity to each other. External charging device 22 and/or IMD 14 may provide one or more audible tones or visual indications of the alignment.

Although inductive coupling is generally described as the method for recharging rechargeable power source 18, other wireless energy transfer techniques may alternatively be used. Any of these techniques may generate heat in IMD 14 such that the charging process may need to be controlled by matching the determined temperature to one or more thresholds, modeling tissue temperatures based on the determined temperature, or using a calculated cumulative thermal dose as feedback.

Recharge module 38 may include one or more circuits that process, filter, convert and/or transform the electrical signal induced in the secondary coil to an electrical signal capable of recharging rechargeable power source 18. For example, in alternating current induction, recharge module 38 may include a half-wave rectifier circuit and/or a full-wave rectifier circuit configured to convert alternating current from the induction to a direct current for rechargeable power source 18. The full-wave rectifier circuit may be more efficient at converting the induced energy for rechargeable power source 18. However, a half-wave rectifier circuit may be used to store energy in rechargeable power source 18 at a slower rate. In some examples, recharge module 38 may include both a full-wave rectifier circuit and a half-wave rectifier circuit such that recharge module 38 may switch between each circuit to control the charging rate of rechargeable power source 18 and temperature of IMD 14.

Rechargeable power source 18 may include one or more capacitors, batteries, and/or other energy storage devices. Rechargeable power source 18 may deliver operating power to the components of IMD 14. In some examples, rechargeable power source 18 may include a power generation circuit to produce the operating power. Rechargeable power source 18 may be configured to operate through many discharge and recharge cycles. Rechargeable power source 18 may also be configured to provide operational power to IMD 14 during the recharge process. In some examples, rechargeable power source 18 may be constructed with materials to reduce the amount of heat generated during charging. In other examples, IMD 14 may be constructed of materials and/or using structures that may help dissipate generated heat at rechargeable power source 18, recharge module 38, and/or secondary coil 40 over a larger surface area of the housing of IMD 14.

Although rechargeable power source 18, recharge module 38, and secondary coil 40 are shown as contained within the housing of IMD 14, in alternative implementations, at least one of these components may be disposed outside of the housing. For example, in some implementations, secondary coil 40 may be disposed outside of the housing of IMD 14 to facilitate better coupling between secondary coil 40 and the primary coil of external charging device 22. These different configurations of IMD 14 components may allow IMD 14 to be implanted in different anatomical spaces or facilitate better inductive coupling alignment between the primary and secondary coils.

IMD 14 may also include temperature sensor 39. Temperature sensor 39 may include one or more temperature sensors configured to measure the temperature of respective portions of IMD 14. As described herein, these temperature sensor(s) may not be thermally coupled to, and may not be directly attached to, the portion of the device for which a temperature is to be determined based on the sensed temperature measured by temperature sensor 39. In one instance, the temperature sensor is not directly attached to the housing 19 or to the exterior surface(s) of housing 19 of the device. In other words, temperature measurement is not performed through direct contact or physical contact between the temperature sensor and the target portion to be measured. Although the temperature sensor may be physically attached to the target portion or target surface through one or more structures, thermal conduction that may occur between the target portion and the sensor is not directly used to measure the temperature of the target portion.

Temperature sensor(s) 39 may include one or more sensors arranged to measure the temperature of a component, surface, or structure, e.g., secondary coil 40, power source 18, recharge module 38, and other circuitry housed within IMD 14. Temperature sensor 39 may be disposed internal of the housing of IMD 14 or otherwise disposed relative to the external portion of housing (e.g., tethered to an external surface of housing via an appendage cord, light pipe, heat pipe, or some other structure). As described herein, temperature sensor 39 may be used to make temperature measurements of internal portions of the IMD 14, the temperature measurements used as a basis for determining the temperature of the housing and/or external surface of IMD 14. For example, processing circuitry 30 or processing circuitry of external charging device 22 may use these temperature measurements to determine the housing/external surface temperatures of IMD 14.

In other examples, temperature measurements may be used to determine temperatures of a specific portion of housing 19 or a component coupled thereto, such as header block 15, or another module that is coupled to IMD 14. For instance, IMD 14 may comprise an additional housing that is separate from, but affixed to, housing 19 that contains some components of IMD 14. As one specific example, a secondary coil such as secondary coil 40 may reside within an additional housing that is external to, but affixed to, main housing 19. Temperature measurements may be used to determine a temperature of a surface or portion of this additional housing or a structure within this housing such as the secondary coil itself. As another example, IMD 14 may carry an appendage protruding from housing 19 carrying one or more electrodes that serves as a stub lead for delivering electrical stimulation therapy. Temperature sensor 39 may be used to make temperature measurements that may be used as a basis for determining the temperature of a portion of this structure. The determined temperatures are then further used as feedback to control the power levels or charge times (e.g., cycle times) used during the charging session of rechargeable power source 18. In some examples, temperature sensor 39 may be used to obtain temperature measurements of a header block 15, or another module that is coupled to IMD 14. For instance, IMD 14 may comprise an additional housing that is separate from, but affixed to, housing 19 that contains some components of IMD 14. As one specific example, a secondary coil may reside within an additional housing. As another example, IMD 14 may carry an appendage protruding from housing 19 carrying one or more electrodes that serves as a stub lead for delivering electrical stimulation therapy. Temperature sensor 39 may be used to make temperature measurements that may be used as a basis for determining the temperature of a surface, or another portion, of these and other structures.

Although a single temperature sensor may be adequate, multiple temperature sensors may provide more specific temperature readings of separate components or of different portions of the IMD. Although processing circuitry 30 may continuously measure temperature using temperature sensor 39, processing circuitry 30 may conserve energy by only measuring temperatures during recharge sessions. Further, temperatures may be sampled at a rate necessary to effectively control the charging session, but the sampling rate may be reduced to conserve power as appropriate. Processing circuitry 30 may be configured to access memory, such as memory 32, to retrieve information comprising instructions, formulas, determined values, and/or one or more constants, and to use this information to execute an algorithm to determine a current temperature, and/or a series of temperatures over time, for the housing 19 and/or exterior surface(s) of housing 19 of IMD 14 based on the measured temperature(s) provided by temperature sensor 39.

Processing circuitry 30 may also control the exchange of information with external charging device 22 and/or an external programmer using telemetry module 36. Telemetry module 36 may be configured for wireless communication using radio frequency protocols, such as BLUETOOTH, or similar RF protocols, as well as using inductive communication protocols. Telemetry module 36 may include one or more antennas 37 configured to communicate with external charging device 22, for example. Processing circuitry 30 may transmit operational information and receive therapy programs or therapy parameter adjustments via telemetry module 36. Also, in some examples, IMD 14 may communicate with other implanted devices, such as stimulators, control devices, or sensors, via telemetry module 36. In addition, telemetry module 36 may be configured to control the exchange of information related to sensed and/or determined temperature data, for example temperatures sensed by and/or determined from temperatures sensed using temperature sensor 39. In some examples, telemetry module 36 may communicate using inductive communication, and in other examples, telemetry module 36 may communicate using RF frequencies separate from the frequencies used for inductive charging.

In some examples, processing circuitry 30 may transmit additional information to external charging device 22 related to the operation of rechargeable power source 18. For example, processing circuitry 30 may use telemetry module 36 to transmit indications that rechargeable power source 18 is completely charged, rechargeable power source 18 is fully discharged, or any other charge status of rechargeable power source 18. In some examples, processing circuitry 30 may use telemetry module 36 to transmit instructions to external charging device 22, including instructions regarding further control of the charging session, for example instructions to lower the power level or to terminate the charging session, based on the determined temperature of the housing/external surface 19 of the IMD.

Processing circuitry 30 may also transmit information to external charging device 22 that indicates any problems or errors with rechargeable power source 18 that may prevent rechargeable power source 18 from providing operational power to the components of IMD 14. In various examples, processing circuitry 30 may receive, through telemetry module 36, instructions for algorithms, including formulas and/or values for constants to be used in the formulas, that may be used to determine the temperature of the housing 19 and/or exterior surface(s) of housing 19 of IMD 14 based on temperatures sensed by temperature sensor 39 located within IMD 14 during and after a recharging session performed on rechargeable power source 18.

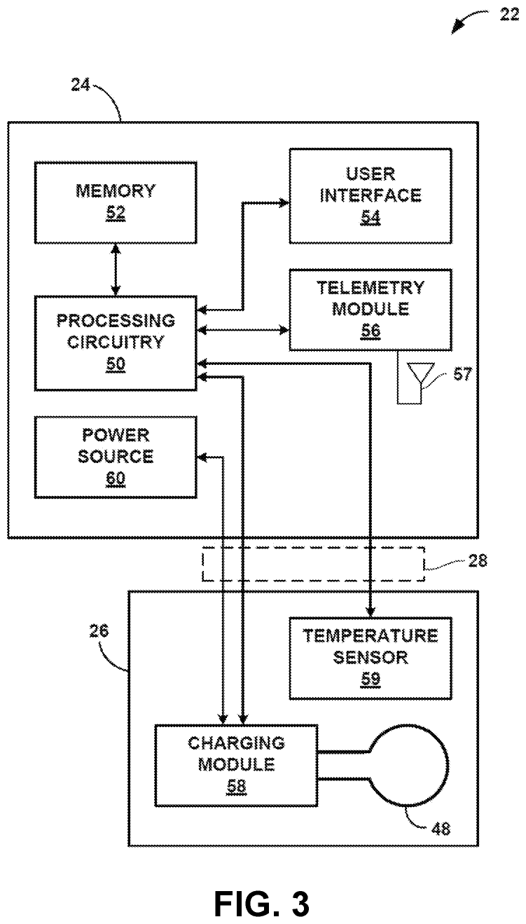

FIG. 3 is a block diagram of an example external charging device 22 of FIG. 1. While external charging device 22 may generally be described as a hand-held device, external charging device 22 may be a larger portable device or a stationary device. In addition, in other examples external charging device 22 may be included as part of an external programmer or include functionality of an external programmer. External charging device 22 may also be configured to communicate with an external programmer. As shown in FIG. 3, external charging device 22 includes two separate components. Housing 24 encloses components such as a processing circuitry 50, memory 52, user interface 54, telemetry module 56, and power source 60. Charging head 26 may include charging module 58, temperature sensor 59, and coil 48. As shown in FIG. 2, housing 24 is electrically coupled to charging head 26 via charging cable 28.

A separate charging head 26 may facilitate optimal positioning of coil 48 over coil 40 of IMD 14. However, charging module 58 and/or coil 48 may be integrated within housing 24 in other examples. Memory 52 may store instructions that, when executed by processing circuitry 50, causes processing circuitry 50 and external charging device 22 to provide the functionality ascribed to external charging device 22 throughout this disclosure, and/or any equivalents thereof.

External charging device 22 may also include one or more temperature sensors, illustrated as temperature sensor 59, similar to temperature sensor 39 of FIG. 2. As shown in FIG. 3, temperature sensor(s) 59 may be disposed within charging head 26. For example, charging head 26 may include one or more temperature sensors positioned and configured to sense the temperature of coil 48 and/or a surface of the housing of charging head 26. In other examples, one or more temperature sensors of temperature sensor 59 may be disposed within housing 24. In some examples, external charging device 22 may not include temperature sensor 59.

In general, external charging device 22 comprises any suitable arrangement of hardware, alone or in combination with software and/or firmware, to perform the techniques ascribed to external charging device 22, and processing circuitry 50, user interface 54, telemetry module 56, and charging module 58 of external charging device 22, and/or any equivalents thereof. In various examples, external charging device 22 may include one or more processors, such as one or more microprocessors, DSPs, ASICs, FPGAs, or any other equivalent integrated or discrete logic circuitry, as well as any combinations of such components. External charging device 22 also, in various examples, may include computer readable storage media, such as memory 52 Memory 52 may be implemented by computer readable storage media such as RAM, ROM, PROM, EPROM, EEPROM, flash memory, a hard disk, a CD-ROM, comprising executable instructions for causing the one or more processors to perform the actions attributed to them. Moreover, although processing circuitry 50, telemetry module 56, charging module 58, and temperature sensor 59 are described as separate modules, in some examples, processing circuitry 50, telemetry module 56, charging module 58, and/or temperature sensor 59 are functionally integrated. In some examples, processing circuitry 50, telemetry module 56, charging module 58, and/or temperature sensor 59 correspond to individual hardware units, such as ASICs, DSPs, FPGAs, or other hardware units. In other examples, one or more functions of external charging device 22 may be combined into a single integrated circuit.

Memory 52 may store instructions that, when executed by processing circuitry 50, cause processing circuitry 50 and external charging device 22 to provide the functionality ascribed to external charging device 22 throughout this disclosure, and/or any equivalents thereof. For example, memory 52 may include instructions that cause processing circuitry 50 to control the power level used to charge IMD 14 in response to the determined system metrics and temperatures for the housing/external surface(s) of IMD 14, as communicated from IMD 14, or instructions for any other functionality. In addition, memory 52 may include a record of selected power levels, sensed temperatures, determined temperatures, or any other data related to charging rechargeable power source 18. Processing circuitry 50 may, when requested, transmit any of this stored data in memory 52 to another computing device for review or further processing. Processing circuitry 50 may be configured to access memory, such as memory 32 of IMD 14 and/or memory 52 of external charging device 22, to retrieve information comprising instructions, formulas, and determined values for one or more constants, and to use this information to perform an algorithm to determine a current temperature, and/or a series of temperatures over time, for the housing 19 and/or exterior surface(s) of housing 19 of IMD 14 based on the measured temperature(s) provided by temperature sensors 39 of IMD 14.

Memory 52 may be configured to store instructions for communication with and/or control of one or more temperature sensors 39 of IMD 14. In various examples, memory 52 stores information related to determining the temperature of the housing 19 and/or exterior surface(s) of housing 19 of IMD 14 based on temperatures sensed by one or more temperature sensors, such as temperature sensors 39, located within IMD 14. For example, memory 52 may store one or more formulas, as further described below, that may be used to determine system metrics and the temperature of the housing 19 and/or exterior surface(s) of housing 19 based on temperature(s) sensed by the temperature sensors 39. Memory 52 may store values for one or more determined constants used by these formulas. Memory 52 may store instructions that, when executed by processing circuitry such as processing circuitry 50, performs an algorithm, including using the formulas, to determine a current temperature, or a series of temperatures over time, for the housing 19 and/or exterior surface(s) of housing 19 of IMD 14 during a charging session and/or for some time after a charging session performed on IMD 14. In some examples, memory 52 may store instructions that, when executed by processing circuitry such as processing circuitry 50, perform an algorithm, including using one or more formulas, to determine a value to be assigned to one or more of the constants used in the algorithm used to determine the temperature(s) associated with the housing 19 and/or exterior surface(s) of housing 19 of IMD 14 during a charging session and/or for some time after a charging session performed on IMD 14.

User interface 54 may include a button or keypad, lights, a speaker for voice commands, a display, such as a liquid crystal (LCD), light-emitting diode (LED), or cathode ray tube (CRT). In some examples, the display may be a touch screen. As discussed in this disclosure, processing circuitry 50 may present and receive information relating to the charging of rechargeable power source 18 via user interface 54. For example, user interface 54 may indicate when charging is occurring, quality of the alignment between coils 40 and 48, the selected power level, current charge level of rechargeable power source 18, duration of the current recharge session, anticipated remaining time of the charging session, sensed temperatures, or any other information. Processing circuitry 50 may receive some of the information displayed on user interface 54 from IMD 14 in some examples. In some examples, user interface 54 may provide an indication to the user regarding the quality of alignment between coils 40, depicted in FIG. 2 and coil 48, based on the charge current to the battery.

User interface 54 may also receive user input via user interface 54. The input may be, for example, in the form of pressing a button on a keypad or selecting an icon from a touch screen. The input may request starting or stopping a recharge session, a desired level of charging, or one or more statistics related to charging rechargeable power source 18 (e.g., the cumulative thermal dose). User input may also include inputs related to temperature thresholds for the IMD that may be used to regulate for example a maximum housing/surface temperature the patient is willing to experience during a charging session of the IMD. The inputs related to threshold values may be store in memory 52, and/or transmitted through telemetry module 56 to IMD 14 for storage in a memory, such as memory 32, located within IMD 14. In this manner, user interface 54 may allow the user to view information related to the charging of rechargeable power source 18 and/or receive charging commands, and to provide inputs related to the charging process. In various examples, user interface 25 as shown and described with respect to FIG. 1 is arranged to perform and to provide the features and/or functions ascribed to user interface 54 as illustrated and described with respect to FIG. 3.

In some examples, user interface 54 may present information related to the power transfer between external charging device 22 and a power receiving unit, e.g., IMD 14 described above in relation to FIG. 1. In some examples, programming instructions at memory 52 may cause processing circuitry 50 to enter a training mode. Training mode may be selectable by a user via user interface 54.

During training mode, a user may change a location and orientation of the power transfer unit relative to the power receiving unit, in some examples, based on a specified pattern displayed on user interface 54. Processing circuitry 50 may determine the location and or orientation that provides a consistent power transfer and output an indication, e.g., via user interface 54 to the user. Processing circuitry 50, or other processing circuitry in system 100, as described above in relation to FIG.1, may calculate the power transfer based on one or more system metrics, such as output efficiency, coupling efficiency, the magnitude of electrical current received by power storage unit 18 depicted in FIG. 2, calculated heating of IMD 14 or other system metrics. User interface 54 may include an audio output, e.g., a tone that changes tone, frequency, pulse repetition or some other audio characteristic as the user changes the relative location or orientation. User interface 54 may also, or alternatively, include a display that changes color, length of a bar on a bar chart, a moving needle, a graph or some other indication of power transfer.

As described above in relation to FIG. 1, in some examples, user interface 54 may display or otherwise describe different patterns that the user should follow to move the primary coil relative to the power receiving unit. In some examples, the user interface may provide an indication of where the primary coil is in a selected pattern, relative to the power receiving unit, or some other location indicator. In this manner, the user interface may track the primary coil path through the provided pattern and display the primary coil location in the path and/or display the portion of the path completed and the portion of the path remaining as different colors, different line widths, different dashed patterns, etc. After gathering system metrics during the relative movement phase of the training mode, the indication displayed by user interface 54 may guide the patient back to a detected “sweet spot.” The sweet spot may not be a single location, but in some examples, a region in which the relative location between the primary coil and secondary coil result in a specified energy transfer, e.g., transfer efficiency above a threshold, INS battery current above a threshold, heating calculation below a threshold, or some other system metric. In some examples, a heating calculation may be an operation performed by processing circuitry of system 100, to estimate the amount of wireless power lost as heat, e.g., by heating tissue surrounding an implantable medical device, such as IMD 14 of FIG. 1, heating the IMD itself, heating of energy transfer coil 26 and so on. In some examples, processing circuitry 50, or other processing circuitry of system 100, may use indications of temperature from one or more temperatures sensors, such as temperature sensor 59, to perform the heating calculation.

In other examples, processing circuitry 50, or some other processing circuitry of system 100 of FIG. 1, may execute programming instructions including a learning algorithm. The learning algorithm may measure and stores system metrics related to power transfer during power transfer sessions over time. In some examples the learning algorithm may record coupling efficiency, or some other measure of power transfer, for a number of power transfer sessions. The learning algorithm may determine a measure of central tendency for the one or more system metrics, such as an average power, median power or some other measure of the power transfer and record the results at memory 52, or some other memory location of system 100. User interface 54 may output an indication to a user, such as patient 12 of FIG. 1, of a relative location and/or relative orientation of charging head 26 and power receiving unit, e.g., IMD 14, that provides a consistent power transfer. In this manner, a patient may learn where to place charging head 26 that result in charging sessions with a predictable duration. Charging head 26 may also be referred to as wand 26.

External charging device 22 also includes components to transmit power to recharge rechargeable power source 18 associated with IMD 14. As shown in FIG. 3, external charging device 22 includes primary coil 48 and charging module 58 coupled to power source 60. Charging module 58 may be configured to generate an electrical current in primary coil 48 from electrical energy stored in or provided by power source 60. Although primary coil 48 is illustrated as a simple loop in FIG. 3, primary coil 48 may include multiple turns of wire. Charging module 58 may generate the electrical current according to a power level selected by processing circuitry 50 based on the sensed and/or determined temperature or temperatures received from IMD 14 and/or a temperature sensor within external charging device 22. As described herein, processing circuitry 50 may select a “high” power level, a “low” power level, or a variety of different power levels to control the rate of recharge in rechargeable power source 18 and the temperature of IMD 14. In some examples, processing circuitry 50 may control charging module 58 based on a power level selected by processing circuitry 30 of IMD 14. The determined temperature of the housing 19 and/or exterior surface(s) of housing 19 of IMD 14 used as feedback for control of the recharge power level may be derived from a temperature sensed by a temperature sensor within IMD 14. Although processing circuitry 50 may control the power level used for charging rechargeable power source 18, charging module 58 may include processing circuitry including one or more processors configured to partially or fully control the power level based on the determined temperatures.

Primary coil 48 may include a coil of wire, e.g., having multiple turns, or other devices capable of inductive coupling with a secondary coil 40 disposed within patient 12. Primary coil 48 may include a winding of wire configured such that an electrical current generated within primary coil 48 can produce a magnetic field configured to induce an electrical current within secondary coil 40. The induced electrical current may then be used to recharge rechargeable power source 18. In this manner, the electrical current may be induced in secondary coil 40 associated with rechargeable power source 18. The coupling efficiency between secondary coil 40 and primary coil 48 of external charging device 22 may be dependent upon the alignment of the two coils. Generally, the coupling efficiency increases when the two coils share a common axis and are in close proximity to each other. User interface 54 of external charging device 22 may provide one or more audible tones or visual indications of the alignment.

Charging module 58 may include one or more circuits that generate an electrical signal, and an electrical current, within primary coil 48. Charging module 58 may generate an alternating current of specified amplitude and frequency in some examples. In other examples, charging module 58 may generate a direct current. In any case, charging module 58 may be capable of generating electrical signals, and subsequent magnetic fields, to transmit various levels of power to IMD 14. In this manner, charging module 58 may be configured to charge rechargeable power source 18 of IMD 14 with the selected power level.

The power level that charging module 58 selects for charging may be used to vary one or more parameters of the electrical signal generated for coil 48. For example, the selected power level may specify wattage, electrical current of primary coil 48 or secondary coil 40, current amplitude, voltage amplitude, pulse rate, pulse width, a cycling rate, or a duty cycle that determines when the primary coil is driven, or any other parameter that may be used to modulate the power transmitted from coil 48. In this manner, each power level may include a specific parameter set that specifies the signal for each power level. Changing from one power level to another power level (e.g., a “high” power level to a lower power level) may include adjusting one or more parameters. For instance, at a “high” power level, the primary coil may be substantially continuously driven, whereas at a lower power level, the primary coil may be intermittently driven such that periodically the coil is not driven for a predetermined time to control heat generation. The parameters of each power level may be selected based on hardware characteristics of external charging device 22 and/or IMD 14.

Power source 60 may deliver operating power to the components of external charging device 22. Power source 60 may also deliver the operating power to drive primary coil 48 during the charging process. Power source 60 may include a battery and a power generation circuit to produce the operating power. In some examples, a battery of power source 60 may be rechargeable to allow extended portable operation. In other examples, power source 60 may draw power from a wired voltage source such as a consumer or commercial power outlet.

External charging device 22 may include one or more temperature sensors shown as temperature sensor 59 (e.g., similar to temperature sensor 39 of IMD 14) for sensing the temperature of a portion of the device. For example, temperature sensor 59 may be disposed within charging head 26 and oriented to sense the temperature of the housing of charging head 26. In another example, temperature sensor 59 may be disposed within charging head 26 and oriented to sense the temperature of charging module 58 and/or coil 48. In other examples, external charging device 22 may include multiple temperature sensors 59 each oriented to any of these portions of device to manage the temperature of the device during charging sessions.

Telemetry module 56 supports wireless communication between IMD 14 and external charging device 22 under the control of processing circuitry 50. Telemetry module 56 may also be configured to communicate with another computing device via wireless communication techniques, or direct communication through a wired connection. In some examples, telemetry module 56 may be substantially similar to telemetry module 36 of IMD 14 described herein, providing wireless communication via an RF or proximal inductive medium. In some examples, telemetry module 56 may include an antenna 57, which may take on a variety of forms, such as an internal or external antenna. Although telemetry modules 56 and 36 may each include dedicated antennas for communications between these devices, telemetry modules 56 and 36 may instead, or additionally, be configured to utilize inductive coupling from coils 40 and 48 to transfer data.

Examples of local wireless communication techniques that may be employed to facilitate communication between external charging device 22 and IMD 14 include radio frequency and/or inductive communication according to any of a variety of standard or proprietary telemetry protocols, or according to other telemetry protocols such as the IEEE 802.11x or Bluetooth specification sets. In this manner, other external devices may be capable of communicating with external charging device 22 without needing to establish a secure wireless connection. As described herein, telemetry module 56 may be configured to receive a signal or data representative of a sensed temperature from IMD 14 or a determined temperature of the housing 19 and/or exterior surface(s) of housing 19 of the IMD based on the sensed temperature. The determined temperature may be determined using an algorithm, including use of formula(s) as further described below, based on measuring the temperature of the internal portion(s) of the IMD, such as circuitry mounted to a circuit board located within IMD 14. In some examples, multiple temperature readings by IMD 14 may be averaged or otherwise used to produce a single temperature value that is transmitted to external charging device 22. The sensed and/or determined temperature may be sampled and/or transmitted by IMD 14 (and received by external charging device 22) at different rates, e.g., on the order of microseconds, milliseconds, seconds, minutes, or even hours. Processing circuitry 50 may then use the received temperature information to control charging of rechargeable power source 18 (e.g., control the charging level used to recharge power source 18).

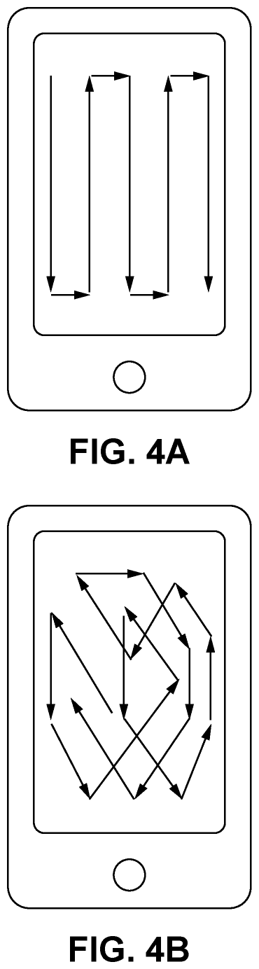

FIGS. 4A and 4B are a conceptual diagrams illustrating a user interface display of example movement patterns for a power transmit unit antenna relative to a power receiving unit during a training mode. For example, FIG. 4A shows a sample up and down pattern (e.g., a non-overlapping serpentine pattern) that may display on the user interface for the user to follow. In another example, FIG. 4B shows a sample criss-cross overlapping movement pattern that a user may follow with the primary coil to determine power transfer efficiency at different relative locations for the power transmitting unit and power receiving unit. Although not shown, the user interface may display start and end locations and/or zoom in on the portion of the pattern that the user should be making as the user moves the primary coil with respect to the power receiving unit.

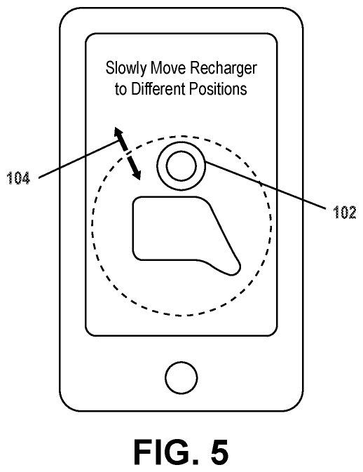

FIG. 5 is a conceptual diagram illustrating an example user interface display for a training mode of a power transmit unit according to one or more techniques of this disclosure. In some examples, the user interface may only provide general instructions to move the primary coil in the region around the power receiving unit. In the example in which the power receiving unit and power transmitting unit communicate with inductive telemetry, the user interface may indicate that the user should stop momentarily at various locations because the power transfer from the power transmitting unit may stop while the devices communicate. In other examples, such as when the devices communicate with RF telemetry and not inductive telemetry, the RF communication may continue while the user moves primary coil to determine the power transfer efficiency of relative positions. In the example of FIG. 5, the user interface may indicate an approximate position 102 of the primary coil as well as the approximate region 104 for placement of the primary coil.

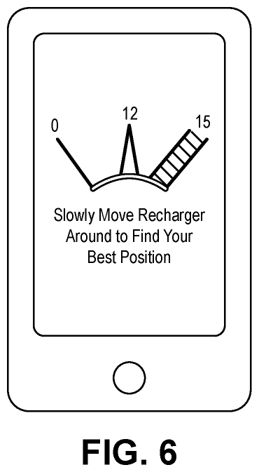

FIG. 6 is a conceptual diagram illustrating an example user interface peak signal display for a training mode of a power transmit unit according to one or more techniques of this disclosure. In the example of FIG. 6, rather than a suggested pattern, as described above in relation to FIGS. 4A-5, the user interface may provide an indication of the current power level or transfer efficiency (12) and a maximum level achieved (15) during the training session. In this manner, the middle triangle shape may be a needle that moves towards the maximum level as the power level increases and toward the zero power level as the power level decreases in real-time as the user moves the primary coil with respect to the power receiving unit.

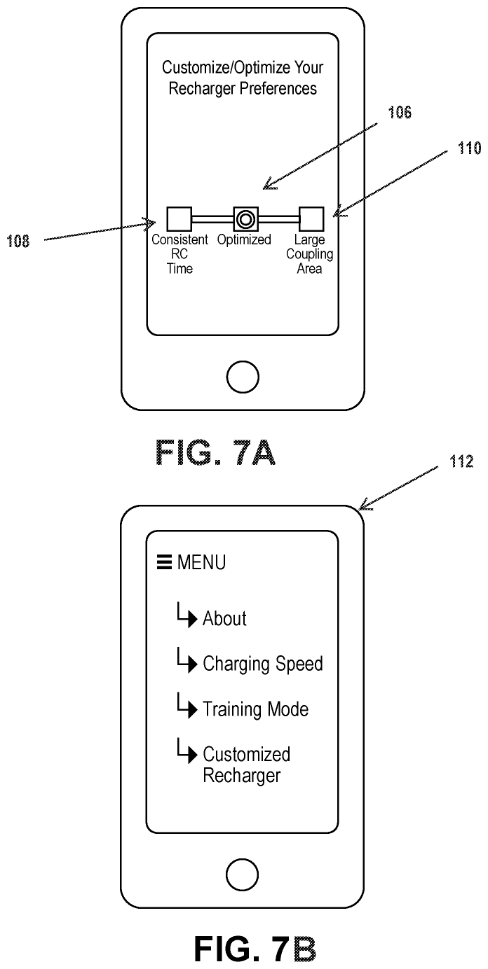

FIGS. 7A and 7B are a conceptual diagram illustrating an example user interface user criteria selection screen for a power transmit unit according to one or more techniques of this disclosure. A user may prefer a shorter charge time, or a user may prefer a larger coupling area. For a shorter charge time, the relative location of the primary and secondary coil may be more constrained to provide the desired power transfer efficiency. A larger area may allow the user to place the primary coil and not be as concerned about relative movement during the charging process. In the example of FIG. 7A, a slider 106, or radio button, may allow the user to select a preference for the charging system, such as between a preference for a consistent recharge time 108 or a large coupling area 110. In some examples, the user may enter the training mode, and user preferences by using a menu 112 displayed on the user interface in FIG. 7B.

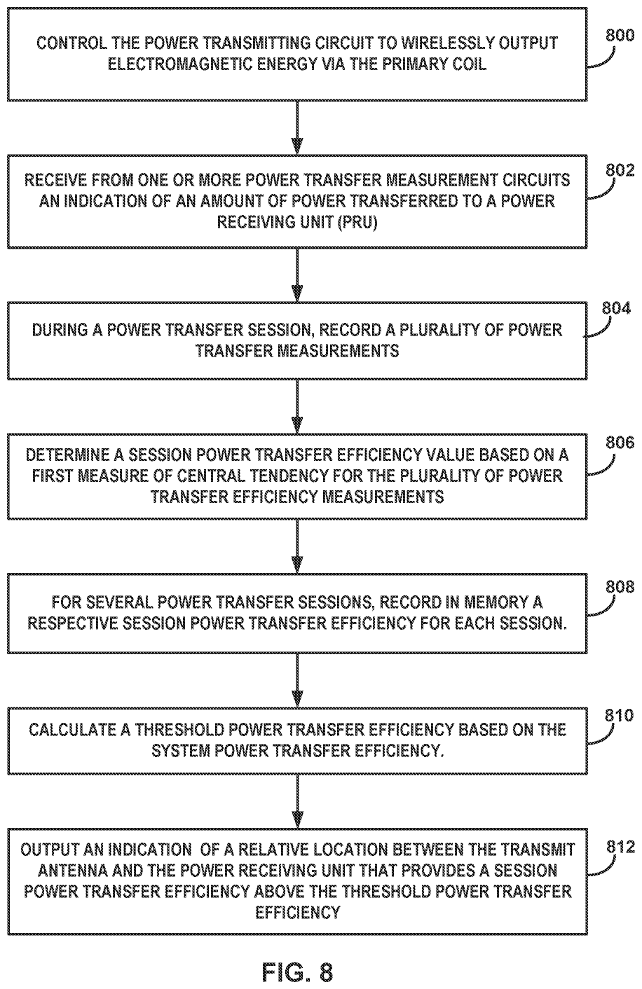

FIG. 8 is a flow diagram illustrating an example operation of the power transfer system of this disclosure. The blocks of FIG. 8 illustrate an example implementation of a learning algorithm, as described above in relation to FIGS. 1 and 3. The functionality of the processing circuitry described below may be executed by any of the processing circuitry in system 100, such as processing circuitry of network computing device 55, processing circuitry 50 of external charging device 22 described above in relation to FIG. 3, or processing circuitry 30 of IMD 14. In some examples each processing circuitry may perform one or more functions and communicate with the other processing circuitry such that steps or portions of steps are shared among the processing circuitry of system 100 to execute the learning algorithm.

Processing circuitry 50 of external charging device 22 may control the power transmitting circuit e.g., charging circuity 56, to wirelessly output electromagnetic energy via primary coil 48 (800). As described above in relation to FIGS. 1 and 3, may also communicate with a power receiving unit, e.g., IMD 14 vi primary coil 48 by inductive communication.

Processing circuitry of system 100, may receive from one or more power transfer measurement circuits an indication of an amount of power transferred to a power receiving unit (PRU), such as IMD 14 (802). In some examples, a power transfer measurement circuit may be located in IMD 14, e.g., to measure Pins_batt and battery current. Power transfer measurement circuits may be located in charging circuitry 56 and elsewhere within external charging device 22. One example of the amount of power transferred may include power transfer efficiency measurements such as INS efficiency, total efficiency and so on, as described above in relation to FIG. 1.

During a power transfer session, processing circuitry may record several power transfer measurements, e.g., at memory 52, or other computer readable storage media of system 100 (804). In some examples, the processing circuitry may record the same type of measurement, e.g., several measurements of battery current, aka charging current, periodically during the power transfer session. In other examples, the processing circuitry may record several measurements over time of different types of power transfer measurements, e.g., different system metrics, e.g., metal loading, Qins, e.g., the amount of heating of IMD 14, and so on. As the implant gets closer and closer, the amount of metal loading on the primary coil will increase. However, sometimes depending on the implant design the highest metal loading location is not the optimal location, for example when there is a large metal portion in the INS such as the header.

In the example in which the processing circuitry records power transfer efficiency, the processing circuitry of system 100 may determine a session power transfer efficiency value based on a first measure of central tendency for the plurality of power transfer efficiency measurements (806). In some examples the measure of central tendency may be an average power transfer efficiency for the session, therefore the processing circuitry executing the learning algorithm may determine the session power transfer efficiency is the average power transfer efficiency of the recorded power transfer efficiency measurements recorded for the session.

Over several power transfer sessions, e.g., over a period of days, weeks and so on, the processing circuitry executing the learning algorithm may record in memory a respective session power transfer efficiency for each session. The processing circuitry may determine a system power transfer efficiency based on a second measure of central tendency for all, or a selected portion, of the recorded session power transfer efficiency values (808). As described above in relation to FIG. 1 measures of central tendency may include average, median, mode and so on. Also as mentioned above, any of the several system metrics may be used for the learning algorithm, either separately or in combination. Power transfer efficiency is just one example implementation.

The processing circuitry, e.g., processing circuitry 50 of FIG. 3, may calculate a threshold power transfer efficiency based on the system power transfer efficiency (810). The processing circuitry may output an indication e.g., via user interface 54, of a relative location between the transmit antenna and the power receiving unit that provides a session power transfer efficiency above the threshold power transfer efficiency (812). Therefore, while the relative location of the transmit antenna, e.g., energy transfer coil 26, is within an area such that the session power transfer efficiency is above the threshold power transfer efficiency, the patient may expect a consistent power transfer. In this manner, a patient may learn where to place energy transfer coil 26 that result in charging sessions with a predictable duration.

The techniques of this disclosure may also be described by the following examples.

### EXAMPLE 1

A system comprising a user interface; power transfer measurement circuitry; a power transmitting circuit comprising a transmit antenna configured to transmit electromagnetic energy to a power receiving device;

processing circuitry operatively coupled to a memory, the processing circuitry configured to: control the power transmitting circuit to wireles sly output the electromagnetic energy to the power receiving device; receive, from the power transfer measurement circuit, an indication of an amount of power transferred to the power receiving device; record a plurality of power transfer measurements; and control the user interface to output an indication of the amount of power transferred, wherein the indication of the amount of power transferred is configured to prompt a user to adjust a position of the transmit antenna relative to the power receiving device based on the plurality of power transfer measurements.

### EXAMPLE 2

The system of example 1, wherein the plurality of power transfer measurements comprises a power transfer efficiency.

### EXAMPLE 3

The system of example 2, wherein the processing circuitry determines the power transfer efficiency based on: a measured value of power received by the power receiving unit; and power at the transmit antenna as well as at a tuning capacitor connected to the transmit antenna.

### EXAMPLE 4

The system of any of examples 1 through 3, wherein the indication of the amount of power transferred is implemented as a graphical display on the user interface.

### EXAMPLE 5

The system of any of examples 1 through 4, wherein the indication of the amount of power transferred is an audible indication output from the user interface.

### EXAMPLE 6

The system of any of examples 1 through 5, wherein the processing circuitry is configured to operate in a training mode; wherein the indication of the amount of power transferred while in the training mode provides a suggested position of the transmit antenna relative to the power receiving device, and wherein the suggested position is based on a user selected criteria.

### EXAMPLE 7

The system of example 6, wherein the user selected criteria comprise criteria selected from at least one category, wherein the at least one category comprises: consistent recharge time, size of power coupling zone, and balance between sweet spot and consistency.

### EXAMPLE 8

The system of any of examples 1 through 7, wherein the power receiving device is an implantable medical device.

### EXAMPLE 9

The system of any of examples 1 through 8, wherein the indication of an amount of power transferred to the power receiving unit comprises one or more of: a digital message from the power receiving unit including a measured value of power received; a digital message from the power receiving unit including a measured value of electrical current received; an estimate of the temperature of one or more portions of the power receiving unit; frequency shift; or metal loading.

### EXAMPLE 10

The system of any of examples 1 through 9, wherein the power transmitting circuit is an inductive power transmitting circuit; and wherein the processing circuitry is configured to cause the power transmitting circuit to pause power transmission while the processing circuitry communicates with the power receiving device.

### EXAMPLE 11

A system comprising a user interface; a power transfer measurement circuit; a power transmitting circuit comprising a transmit antenna; processing circuitry operatively coupled to a memory, the processing circuitry configured to: control the power transmitting circuit to wirelessly output electromagnetic energy; receive from the power transfer measurement circuit an indication of an amount of power transferred to a power receiving unit (PRU); during a power transfer session, record a plurality of power transfer efficiency measurements; determine a session power transfer efficiency value based on a first measure of central tendency for the plurality of power transfer efficiency measurements; determine a system power transfer efficiency based on a second measure of central tendency for a plurality of session power transfer efficiency values; calculate a threshold power transfer efficiency based on the system power transfer efficiency; and output an indication via the user interface of a relative location between the transmit antenna and the power receiving unit that provides a session power transfer efficiency above the threshold power transfer efficiency.

### EXAMPLE 12

The system of example 11, wherein the threshold power transfer efficiency is further based on a user selected criteria.

### EXAMPLE 13

The system of any of examples 11 and 12, wherein the user selected criteria comprise criteria selected from at least one category, wherein the at least one category comprises: “consistent recharge time,” “size of power coupling zone,” or “optimized sweet spot and consistency.”.

### EXAMPLE 14

A method includes controlling, by processing circuitry operatively coupled to a memory, a power transmitting circuit to wireles sly output electromagnetic energy to power receiving device, wherein the power transmitting circuit comprises a transmit antenna configured to output the electromagnetic energy to the power receiving device ; receiving, by processing circuitry and from a power transfer measurement circuit, an indication of an amount of power transferred to the power receiving device; recording, by processing circuitry, a plurality of power transfer measurements; controlling, by the processing circuitry, a user interface to output an indication of the amount of power transferred, wherein the indication of the amount of power transferred is configured to prompt a user to adjust a position of the transmit antenna relative to the power receiving device based on the plurality of power transfer measurements.

### EXAMPLE 15

The method of example 14, wherein the plurality of power transfer measurements comprises a power transfer efficiency, and wherein the processing circuitry determines the power transfer efficiency based on: a measured value of power received by the power receiving unit; and power in the transmit antenna as well as at a tuning capacitor connected to the transmit antenna.

### EXAMPLE 16

The method of any of examples 14 and 15, wherein the indication of the amount of power transferred is implemented as a graphical display on the user interface.

### EXAMPLE 17

The method of any of examples 14 through 16, wherein the indication of the amount of power transferred is an audible indication output from the user interface.

### EXAMPLE 18

The method of any of examples 14 through 17, further comprising operating, by the processing circuitry, in a training mode, wherein the indication of the amount of power transferred while in the training mode provides a suggested position of the transmit antenna relative to the power receiving device, and wherein the suggested position is based on a user selected criteria.

### EXAMPLE 19

The method of any of examples 14 through 18, wherein the indication of an amount of power transferred to the power receiving unit comprises one or more of: a digital message from the power receiving unit including a measured value of power received; a digital message from the power receiving unit including a measured value of electrical current received; an estimate of the temperature of one or more portions of the power receiving unit; frequency shift; or metal loading.

### EXAMPLE 20

The method of any of examples 14 through 19, wherein the power transmitting circuit is an inductive power transmitting circuit, the method further comprising, controlling, by the processing circuitry, the power transmitting circuit to pause power transmission while the processing circuitry communicates with the power receiving device.

In one or more examples, the functions described above may be implemented in hardware, software, firmware, or any combination thereof. For example, the various components of FIGS. 1-3, such as charging device 24, external computing device 25, network computing device 55, processing circuitry 30, and processing circuitry 50 may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

The term “non-transitory” may indicate that the storage medium is not embodied in a carrier wave or a propagated signal. In certain examples, a non-transitory storage medium may store data that can, over time, change (e.g., in RAM or cache). By way of example, and not limitation, such computer-readable storage media, may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Combinations of the above should also be included within the scope of computer-readable media.

Instructions may be executed by one or more processors, such as one or more DSPs, general purpose microprocessors, ASICs, FPGAs, or other equivalent integrated or discrete logic circuitry. Accordingly, the term “processor” and “processing circuitry,” as used herein, such as processing circuitry 30, may refer to any of the foregoing structure or any other structure suitable for implementation of the techniques described herein. Also, the techniques could be fully implemented in one or more circuits or logic elements.

The techniques of this disclosure may be implemented in a wide variety of devices or apparatuses, including, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples of the disclosure have been described. These and other examples are within the scope of the following claims.

What is claimed is:

1. A system comprising:
a user interface;
power transfer measurement circuitry;
a power transmitting circuit comprising a transmit antenna configured to transmit electromagnetic energy to a power receiving device;
processing circuitry operatively coupled to a memory, the processing circuitry configured to:
control the power transmitting circuit to wireles sly output the electromagnetic energy to the power receiving device;
receive, from the power transfer measurement circuit, an indication of an amount of power transferred to the power receiving device;
record a plurality of power transfer measurements; and
control the user interface to output an indication of the amount of power transferred, wherein the indication of the amount of power transferred is configured to prompt a user to adjust a position of the transmit antenna relative to the power receiving device based on the plurality of power transfer measurements.

2. The system of claim 1, wherein the plurality of power transfer measurements comprises a power transfer efficiency.

3. The system of claim 2, wherein the processing circuitry determines the power transfer efficiency based on:
a measured value of power received by the power receiving unit; and
power at the transmit antenna as well as at a tuning capacitor connected to the transmit antenna.

4. The system of claim 1, wherein the indication of the amount of power transferred is implemented as a graphical display on the user interface.

5. The system of claim 1, wherein the indication of the amount of power transferred is an audible indication output from the user interface.

6. The system of claim 1,
wherein the processing circuitry is configured to operate in a training mode;
wherein the indication of the amount of power transferred while in the training mode provides a suggested position of the transmit antenna relative to the power receiving device, and
wherein the suggested position is based on a user selected criteria.

7. The system of claim 6, wherein the user selected criteria comprise criteria selected from at least one category, wherein the at least one category comprises: consistent recharge time, size of power coupling zone, and balance between sweet spot and consistency.

8. The system of claim 1, wherein the power receiving device is an implantable medical device.

9. The system of claim 1, wherein the indication of an amount of power transferred to the power receiving unit comprises one or more of:
a digital message from the power receiving unit including a measured value of power received;
a digital message from the power receiving unit including a measured value of electrical current received;
an amount of heating of the power receiving unit;
an estimate of the temperature of one or more portions of the power receiving unit;
frequency shift; or
metal loading.

10. The system of claim 1,
wherein the power transmitting circuit is an inductive power transmitting circuit; and
wherein the processing circuitry is configured to cause the power transmitting circuit to pause power transmission while the processing circuitry communicates with the power receiving device.

11. A system comprising:
a user interface;
a power transfer measurement circuit;
a power transmitting circuit comprising a transmit antenna;
processing circuitry operatively coupled to a memory, the processing circuitry configured to:
control the power transmitting circuit to wireles sly output electromagnetic energy;
receive from the power transfer measurement circuit an indication of an amount of power transferred to a power receiving unit (PRU);
during a power transfer session, record a plurality of power transfer efficiency measurements;
determine a session power transfer efficiency value based on a first measure of central tendency for the plurality of power transfer efficiency measurements;
determine a system power transfer efficiency based on a second measure of central tendency for a plurality of session power transfer efficiency values;
calculate a threshold power transfer efficiency based on the system power transfer efficiency; and
output an indication via the user interface of a relative location between the transmit antenna and the power receiving unit that provides a session power transfer efficiency above the threshold power transfer efficiency.

12. The system of claim 11, wherein the threshold power transfer efficiency is further based on a user selected criteria.

13. The system of claim 11, wherein the user selected criteria comprise criteria selected from at least one category, wherein the at least one category comprises:
“consistent recharge time,” “size of power coupling zone,” or “optimized sweet spot and consistency.”

14. A method comprising:
controlling, by processing circuitry operatively coupled to a memory, a power transmitting circuit to wirelessly output electromagnetic energy to power receiving device, wherein the power transmitting circuit comprises a transmit antenna configured to output the electromagnetic energy to the power receiving device;
receiving, by the processing circuitry and from a power transfer measurement circuit, an indication of an amount of power transferred to the power receiving device;
recording, by the processing circuitry, a plurality of power transfer measurements;
controlling, by the processing circuitry, a user interface to output an indication of the amount of power transferred, wherein the indication of the amount of power transferred is configured to prompt a user to adjust a position of the transmit antenna relative to the power receiving device based on the plurality of power transfer measurements.

15. The method of claim 14,
wherein the plurality of power transfer measurements comprises a power transfer efficiency, and
wherein the processing circuitry determines the power transfer efficiency based on:
a measured value of power received by the power receiving unit; and
power in the transmit antenna as well as at a tuning capacitor connected to the transmit antenna.

16. The method of claim 14, wherein the indication of the amount of power transferred is implemented as a graphical display on the user interface.

17. The method of claim 14, wherein the indication of the amount of power transferred is an audible indication output from the user interface.

18. The method of claim 14, further comprising operating, by the processing circuitry, in a training mode,
wherein the indication of the amount of power transferred while in the training mode provides a suggested position of the transmit antenna relative to the power receiving device, and
wherein the suggested position is based on a user selected criteria.

19. The method of claim 14, wherein the indication of an amount of power transferred to the power receiving unit comprises one or more of:
a digital message from the power receiving unit including a measured value of power received;
a digital message from the power receiving unit including a measured value of electrical current received;
an amount of heating of the power receiving unit;
an estimate of the temperature of one or more portions of the power receiving unit;
frequency shift; or
metal loading.

20. The method of claim 14, wherein the power transmitting circuit is an inductive power transmitting circuit, the method further comprising, controlling, by the processing circuitry, the power transmitting circuit to pause power transmission while the processing circuitry communicates with the power receiving device.
